(19) United States
(12) Patent Application Publication
(10) Pub. No.: US 2005/0227221 A1
(43) Pub. Date: Oct. 13, 2005

(54) Methods and systems for evaluating and for comparing methods of testing tissue samples

(76) Inventor: James M. Minor, Los Altos, CA (US)

Correspondence Address: AGILENT TECHNOLOGIES, INC.;INTELLECTUAL PROPERTY ADMINISTRATION, LEGAL DEPT., P.O. BOX 7599, M/S DL429, LOVELAND, CO, 80537-0599, US

(21) Appl. No.: 10/821,829

(22) Filed: Apr. 9, 2004

(51) Int. Cl. C12Q 1/00; G06F 19/00; G01N 33/48; G01N 33/50

(52) U.S. Cl. 435/4; 702/19

(57) ABSTRACT

Using contextual response profiles to group genes into “equivalent” classes to infer biological functionality. Comparing such classes from different contexts (conditions) to identify genes that change functionality. Also, methods, systems and computer readable media to separate gene expression signatures and distinguish differential gene expression specific to pure tissue in a heterogeneous tissue sample. Further, methods, systems and computer readable media for validating or calibrating a plotted curve of sorted p-values is provided. Still further, methods, systems and computer readable media are provided for distinguishing differentially expressed genes based on plotting expression levels and replicates derived from one or more genes in a first sample against corresponding expression levels and replicated derived from one or more genes in a second sample.

### BACKGROUND OF THE INVENTION

Cells from different tissues are specialized for performing different functions in an organism. Although it is not known just what makes one cell function as smooth muscle, another as a neuron, and still another as prostate, a cell's function is enabled by the proteins it produces, which in turn depends on its expressed genes.

A gene expression profile over a number of genes is referred to as “gene expression signature.” A gene expression signature, as the name implies, often can signature certain events of the cell, such as disease or toxicological responses. Each toxicological response, for example, can create a specific gene signature. Thus, if it is unknown what toxicological agent is affecting the cell, the measured gene signature of the cell can be compared to library of gene signatures in an effort to identify a match to a known corresponding toxicological agent. Thus, the gene expression signature has become an important subject for biologists. Referred to as “response expression signature”, another type of signature is created by the expression of a specific gene over a series of conditions, e.g., a series composed of designed, controlled, and/or identifiable conditions. Associations among such signatures imply important multi-gene activities and interactions. For example if a subset of such profiles trend/synchronize together, that gene subset may be grouped within a biologically meaningful activity. Also, given another series of different conditions, the profile subsets may be similar except that some genes may change their membership to a different profile subset. Such genes have likely altered their functionality and are candidates for the set of biologically important genes known as functional variants. Examples include SNPs (single nucleotide polymorphisms), splice variants, transcription factors, and any other possibly unrealized form of altering a gene's function to address different conditions of cellular exposure.

One common problem in present biological studies of gene expression signature is that a sample of pure tissue cannot be easily separated from an inherently heterogeneous tissue sample. An example of the problem is that, in order to study the gene expression signatures relevant to the disease process in a glial cell tumor, the glial cells, where particularly the diseased glial cells need to be separated from “normal” glial cells, as well other brain cells/tissue. However, it is difficult, if not impossible, to separate glial cells from the other cells, and as a result, the gene expression signatures relevant to the activity of the tumorous glial cells are convolved with those of irrelevant material that is inherently in the sample being examined. Consequently, the measured gene expression signature of glial tumor may include contribution of the brain cells, as well as of normal (non-tumor) glial cells. Thus, for proper analysis of a heterogeneous sample having a natural mixture of various cells, there is a need for methods to separate gene expression signatures and distinguish differential gene expression specific to each pure tissue in the heterogeneous tissue sample, enabled by response expression signatures over known changing conditions of cell densities. Such need is met by the present invention, as described below.

Another problem in biological studies of gene expression signature is that existing methods for processing gene expression levels cannot be evaluated easily. For example, when using microarray techniques, there are several methods for signal processing to determine gene expression levels and find significant effects. However, evaluation of the capabilities of such methods cannot be easily performed. Thus, there is also a need for methods to evaluate and rank the existing techniques for processing gene expression levels.

### SUMMARY OF THE INVENTION

The present invention provides methods, systems and computer readable media for statistically evaluating characteristic signatures characterizing at least two different types of samples present in a heterogeneous mixture of the samples, to identify one of the types based upon a known or expected trend line characterizing density or activity of that type of sample across a heterogeneous region from which the samples are taken.

According to one aspect of the present invention, methods, systems and computer readable media are provided for rank ordering characteristic signatures of cell properties, by analyzing a heterogeneous tissue region provided with a first portion of the heterogeneous tissue region having at least first and second types of tissue and being bordered by a second portion of the of samples, and a plurality of characteristic signatures are formed using the measured plurality of properties, each of the characteristic signatures characterizing one of the plurality of properties, respectively. A trend profile of cell activity for the second type of tissue along the determined profile of locations through the heterogeneous tissue region is provided, and statistical analysis is conducted on each of the plurality of characteristic signatures with regard to the provided trend profile. The plurality of characteristic signatures are then rank-ordered based on proximity to the trend profile as determined by the statistical analysis.

Further disclosed are methods, systems and computer readable media for validating/calibrating a plotted curve of sorted p-values against the ranks of the p-values based on the order of the sorted p-values, wherein the p-values are calculated with regard to characteristic signature profiles each generated from a plurality of property values from a plurality of samples, and wherein each said p-value, as statistically calculated, represents the probability that the corresponding characteristic signature profile does not match a predefined signature profile.

Methods, systems and computer readable media are provided for distinguishing differentially-expressed genes based plotting one set of expression level values against another set of corresponding expression level values, and including plotting an expression level of each of one or more genes for a first sample against an expression level for each of the same one or more genes in a second sample; plotting one or more replicates of the expression levels; and determining whether a particular gene from a first sample is differentially expressed relative to the same gene from the second sample, based upon the values of the measured expression levels and their replicates for the particular gene.

These and other advantages and features of the invention will become apparent to those persons skilled in the art upon reading the details of the invention as more fully described below.

### BRIEF DESCRIPTION OF THE DRAWINGS

FIG. 1 shows a conventional heterogeneous tissue region including healthy and diseased tissue with an arrow to indicate the locations where a plurality of samples are taken.

FIG. 2 shows a heterogeneous tissue region and an expected profile of activity or density of diseased tissue, when it is considered or known that the center of mass or highest activity of the diseased tissue is at the center of the tissue region.

FIG. 3 shows a heterogeneous tissue region and an expected profile of activity or density of diseased tissue, when it is considered or known that the center of mass or highest activity of the diseased tissue is at the periphery of the tissue region.

FIG. 4 shows distribution of gene expression levels and the known or expected trend of disease-gene activity along a direction in accordance with one embodiment of the present teachings.

FIG. 5 is a flow chart illustrating an example approach toward identifying genes that are related to, or active in a disease process or other anomaly being studied.

FIG. 6 is a pCurve™ for a mixture dilution trends in accordance with the teachings of the present invention.

FIG. 7 is a flow chart illustrating an example of steps that may be taken to generate a pCurve™ such as shown in FIG. 6.

FIG. 8A shows an example of a T-chart that may be used to identify significantly expressed genes using clone groups.

FIG. 8B shows a conventional chart of genes from one experiment plotted against the same genes from another experiment.

FIG. 8C, in comparison shows the same experimental data from FIG. 8B, having been plotted in a T-chart, according to the present invention, after taking noise factors into consideration.

FIG. 9 is a flow chart illustrating steps that may be taken to distinguish differentially-expressed genes using the T-chart of FIG. 8A in accordance with one embodiment of the present teachings.

FIG. 10 is a block diagram illustrating an example of a generic computer system that may be used in implementing the present invention.

### DETAILED DESCRIPTION OF THE INVENTION

Before the present methods and systems are described, it is to be understood that this invention is not limited to particular diseases, heterogeneous samples, methods, method steps or statistical methods, hardware or software described, as such may, of course, vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated by reference to disclose and describe the methods and/or materials in connection with which the publications are cited.

It must be noted that, as used herein and in the appended claims, the singular forms “a”, “and”, and “the” include plural referents unless the context clearly dictates otherwise. Thus, for example, reference to “a sample” includes a plurality of such samples and reference to “the microarray” includes reference to one or more microarrays and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DEFINITIONS

A “pCurve™” as used herein, refers to a sorted p-value profile of a series of statistical, hypothesis-driven evaluations.

A “T-chart”, as used herein refers to data re-plotted by coordinates, scaled in terms of noise units, so that statistical significance is more readily visually apparent.

A “microarray”, “bioarray” or “array”, unless a contrary intention appears, includes any one-, two-or three-dimensional arrangement of addressable regions bearing a particular chemical moiety or moieties associated with that region. A microarray is “addressable” in that it has multiple regions of moieties such that a region at a particular predetermined location on the microarray will detect a particular target or class of targets (although a feature may incidentally detect non-targets of that feature). Array features are typically, but need not be, separated by intervening spaces. In the case of an array, the “target” will be referenced as a moiety in a mobile phase, to be detected by probes, which are bound to the substrate at the various regions. However, either of the “target” or “target probes” may be the one, which is to be evaluated by the other.

Typically a “pulse jet” is a device which can dispense drops in the formation of an array. Pulse jets operate by delivering a pulse of pressure to liquid adjacent an outlet or orifice such that a drop will be dispensed therefrom. Any given substrate may carry one, or more arrays disposed on a front surface of the substrate. A typical array may contain more than ten, more than one hundred, more than one thousand, more than ten thousand features, or even more that one hundred thousand features, in an area of less that 20 cm² or even less that 10 cm². For example, features may have widths in the range from about 10 μm to 1.0 cm. In other embodiments, each feature may have a width (that is, diameter for a round spot) in the range of about 1.0 μm to 1.0 mm, and more usually about 10 μm to 200 μm. Non-round features may have area ranges equivalent to that of circular features with the foregoing with ranges. At least some, or all, of the features are of different compositions, each feature typically being of a homogeneous composition within the feature. Interfeature areas will typically be present which do not carry chemical moiety of a type of which the features are composed. Such interfeature areas typically will be present where the arrays are formed by processes involving drop deposition of reagents but may not be present when, for example, photolithographic array fabrication processes are used. It will be appreciated though, that the interfeature areas, when present, could be of various sizes and configurations. Methods to fabricate arrays are described in detail in U.S. Pat. Nos. 6,242,266; 6,232,072; 6,180,351; 6,171,797 and 6,323,043. As already mentioned, these references are incorporated herein by reference. Other drop deposition methods can be used for fabrication, as previously described herein. Also, instead of drop deposition methods, photolithographic array fabrication methods may be used. Interfeature areas need not be present particularly when the arrays are made by photolithographic methods as described in those patents.

Following receipt by a user, an array will typically be exposed to a sample and then read. Reading of an array may be accomplished by illuminating the array and reading the location and intensity of resulting fluorescence at multiple regions on each feature of the array. For example, a scanner may be used for this purpose is the AGILENT MICROARRAY SCANNER manufactured by Agilent Technologies, Palo, Alto, Calif. or other similar scanner. Other suitable apparatus and methods are described in U.S. Pat. Nos. 6,518,556; 6,486,457; 6,406,849; 6,371,370; 6,355,921; 6,320,196; 6,251,685 and 6,222,664. However, arrays may be read by any other methods or apparatus than the foregoing, other reading method including other optical techniques or electrical techniques (where each feature is provided with an electrode to detect bonding at that feature in a manner disclosed in U.S. Pat. Nos. 6,251,685, 6,221,583 and elsewhere).

A “gene expression signature” or “gene expression profile”, refers to a gene expression profile over a number of genes, typically from the same sample, which may include all of the genes being measured for that sample, or a selected number of those genes. Specific gene expression signatures can often identify specific events occurring within a cell.

A “gene expression response signature” or “gene expression response profile” refers to a profile generated by expression values of the same gene over a number of samples.

When one item is indicated as being “remote” from another, this is referenced that the two items are at least in different buildings, and may be at least one mile, ten miles, or at least one hundred miles apart.

“Communicating” information references transmitting the data representing that information as electrical signals over a suitable communication channel (for example, a private or public network).

“Forwarding” an item refers to any means of getting that item from one location to the next, whether by physically transporting that item or otherwise (where that is possible) and includes, at least in the case of data, physically transporting a medium carrying the data or communicating the data.

A “processor” references any hardware and/or software combination which will perform the functions required of it. For example, any processor herein may be a programmable digital microprocessor such as available in the form of a mainframe, server, or personal computer. Where the processor is programmable, suitable programming can be communicated from a remote location to the processor, or previously saved in a computer program product. For example, a magnetic or optical disk may carry the programming, and can be read by a suitable disk reader communicating with each processor at its corresponding station.

Reference to a singular item, includes the possibility that there are plural of the same items present.

“May” means optionally.

Methods recited herein may be carried out in any order of the recited events which is logically possible, as well as the recited order of events.

All patents and other references cited in this application, are incorporated into this application by reference except insofar as they may conflict with those of the present application (in which case the present application prevails).

One common problem in the preparation of biological samples to be studied, tested, etc., is that sometimes the preparer cannot obtain pure, homogeneous samples of biological material to be studied or tested. An example of this occurs in the study of brain cancer, and specifically where researchers are tying to study tumor tissue in the glial cells. In this situation it is very difficult, if not impossible, to separate the glial cells from the remaining brain tissue. This is just one example among many, where it is difficult, if not impossible, to get a pure sample to study/test. Other examples include attempts to identify functionally variant genes, the functions of which vary under different conditions, as well as toxicity studies, wherein effects on different tissue/genes are desired to be identified, and also in drug discovery processes, where it is desired to know the targets or effects of different drugs on different genes or tissues. A further discussion of drug discovery examples may be found in co-pending, commonly owned application Ser. No. 10/640,081 filed Aug. 13, 2003 and titled “Methods and System for Multi-Drug Treatment Discovery” which is hereby incorporated herein, in its entirety, by reference thereto. Still further, the identification of a homogeneous substance, material or property may be desired from a heterogeneous mixture of substances, materials or properties, such as occurs in mass spectrometry studies, as one example. When a heterogeneous mixture of substances, such as cells is provided to a researcher, this “muddies the waters” considerably in regard to any measurements or characterizations that the researcher may be trying to obtain with respect to a homogeneous member of the heterogeneous mixtures (such as when trying to separate/identify cancer cells from non-cancerous cells, for example), since the researcher is in fact looking at a mixture or combination of the various homogeneous components that make up the heterogeneous mixture (e.g., cancerous cells and non-cancerous cells, some of which may not even be cells of the same origin).

In these situations, when attempting to study any characteristics of the target material (in this case, cancer cells), the characteristics of the other materials are convolved with those of the target material, making it difficult to obtain meaningful data. For example, a researcher interested in studying genetic profiles of the cancer cells is faced with a difficult task because the gene expression signatures of the cancer cells are convolved with the gene expression signatures of the cells, which are non-cancerous.

The present invention addresses these problems by correlating trends of the measured features from samples extending across a target region to be studied, and including samples outside of the target region to be studied, with the expected distribution of the target material of interest in the target region. When working with cells, for example, biologists with experience relating to the particular cells of interest generally know where the active regions are in the target region of interest. Analysis or quantification of the samples may be performed by any applicable analysis method, including microarray/gene expression analysis, protein abundance analysis, mass spectrometry, gas chromatograph, etc., even though the examples described herein focus on gene expression analysis. The analysis results of the samples are arranged in the order of the samples from which they were taken, and then trends in the analysis results are looked for which follow the trend(s)/expected trend(s) of the target material across the same order.

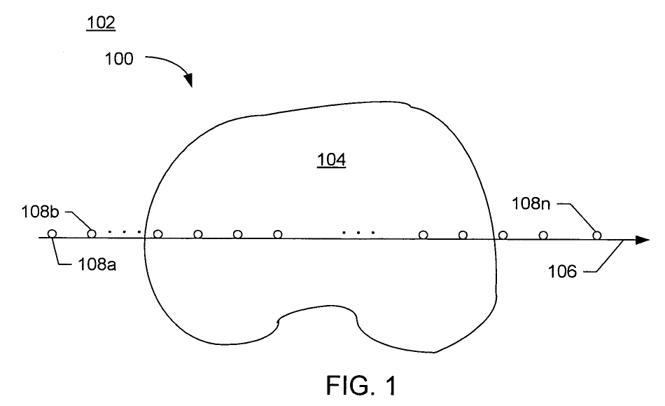

As indicated one example of application of the present invention involves taking tissue samples across a target location that contains tissues of interest to be studied. For example, FIG. 1 illustrates heterogeneous tissue sample 100 which includes a target location or region 104 containing tissues or cells of interest (such as cancer cells, for example) and outlying tissues 102 where it is relatively certain that none (or insignificant amounts) of the cells of interest exist. Hereinafter, for simplicity, the two regions of tissue 102 and 104 are referred to as healthy and diseased tissue, respectively. However, as already noted previously, the “diseased tissue” 104 does not consist purely of diseased cells, but is a combination of the cells of interest (diseased cells) and non-diseased cells, which may include cells of the same type as the diseased cells as well as cells of different types. The examples shown illustrate the present techniques in one dimension for sake of simplicity. However, the same techniques and approaches may readily be extended to two or more dimensional analysis. A series of samples 108a, 108b, . . . , 108n are taken along a line 106 which extends through the center of diseased region 104 and into healthy regions 102 on both sides. Alternatively, a series of samples may be taken along any trajectory through disease region 104 where expected changes in density of diseased tissues can be predicted or hypothesized. A line through the center of diseased region 104 is typically chosen to characterize the expected profile of the diseased cells, although the current techniques are not limited to this line. If there is greater knowledge about the diseased tissue behavior/activity/existence along some other line, then it would make sense to take samples along that line.

Also, for this one-dimensional example, the tissue samples 108a, 108b, . . . 108n are all taken at the same depth (direction into the page) which will typically be the depth where the center of the diseased tissue 104 is located so that the trajectory design creates density variation in disease-specific tissue. Of course, two dimensional analyses may be conducted by taking samples along a line perpendicular to line 106 as well. Additionally, or alternatively, a series of one-dimensional analyses may be conducted along a series of such lines 106 which differ from one another, and then used for relevance studies and/or as replicate information. Typically, at least the samples 108a and 108n are reference samples taken from a location remote from the diseased tissue 104, to act as a “baseline” for normal tissue readings relative to the diseased tissue readings. The interval between neighboring locations for the samples 108 may be determined considering spatial resolution of samples.

For each of the non-diseased samples 108 taken from the heterogeneous tissue sample 100, analysis measurements (such as gene expression levels, for example) may be established. In one non-limiting example of the present teachings, measurement of gene expression levels may be performed using microarray techniques. In such an example, a reference sample, such as 108a or 108n, and a diseased sample may be prepared on a single two-color microarray. In another embodiment, the reference and diseased samples may be prepared on two single-color microarrays, and then compared to determine differential expression values. In both embodiments, the prepared samples may be fluorescently labeled and the reading of the microarray for a gene may be accomplished by illuminating the microarray to produce fluorescence at multiple regions on each feature of the microarray. Hereinafter, microarray techniques are understood to be the techniques used for establishing gene expression level measurements and for determining differential expression values. However, it should be apparent to one of ordinary skill in the art that measurements can also be performed using any other suitable methodologies.

Two channel or two color microarray methods provide a specific advantage for specific comparisons of one tissue to another, but can also enable universal comparisons via a reference sample. Use of two arrays to provide ratios is an inherently more complex process than using only one. Each time an array is run, there is inherent noise associated with the measurements at each probe. Noise values are random and change each time an array is run. However, when both samples are run on a two channel array, then these noise values cancel out when calculating differential values, since the noise level is about the same and correlated for both colors, both being on the same array. However, the single channel technique may be more convenient in the sense that the reference sample need be processed only once, and can then be compared against each of the other samples having been run on a single channel array. However, the reference sample in this instance is an external reference. In contrast, the two color microarray method provides an internal reference, which is inherently safer and more reliable, and the biological preparation noise is eliminated, as discussed above.

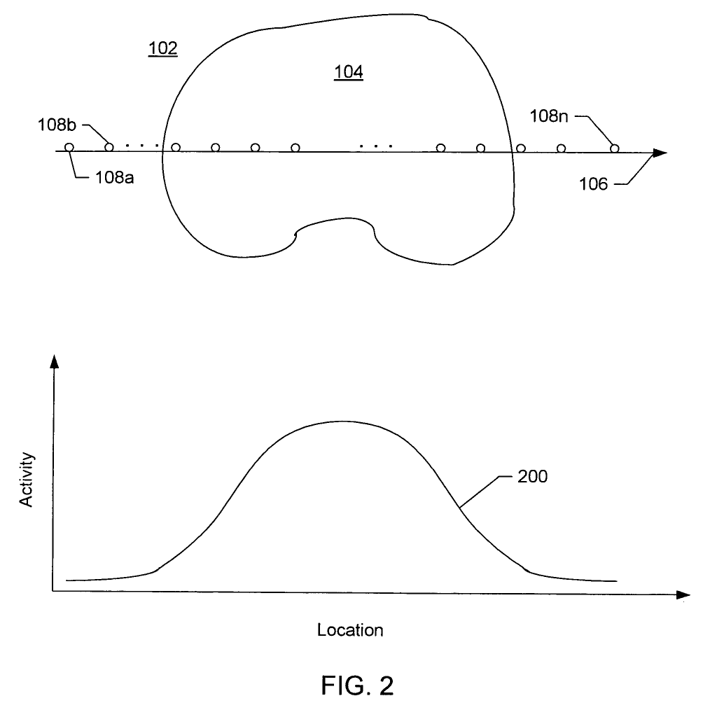

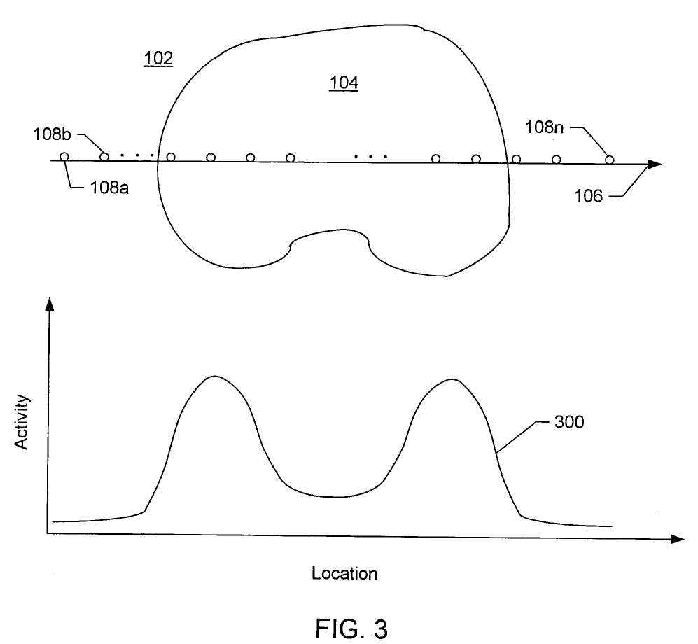

The activity of the diseased tissue is generally proportional to the percentage of the tissue at any given location that is taken up by the diseased tissue versus the non-diseased or healthy tissue. Biologists studying a tissue anomaly of interest are generally aware of where the activity of a tumor or other target region is concentrated. Thus, for example, if the density or highest activity of a target region is in the center of a target region, then genes which are active in, related to, or affected by the disease process will produce a signature that corresponds to the activity or density profile of the diseased tissue. For example, FIG. 2 shows a profile 200 for activity or density of diseased tissue relative to the samples taken by locations 108a, . . . , 108n, where the density or activity is greatest at the center of region 104. However, activity or density may be greatest in locations other than the center of the target region. For example, FIG. 3 shows a profile 300 for activity or density of diseased tissue relative to the samples taken by locations 108a, . . . , 108n, where the density or activity is greatest at the periphery or borders of the tumor 104.

For each tissue sample 108a, 108b, . . . , 108n taken, measurements of the tissue are taken, such as gene expression values, for example. For microarray applications, at least one microarray is run for each tissue sample 108a, 108b, . . . , 108n, and differential expression levels of the genes for each sample are calculated by comparison with a reference, such as sample 108a or 108n, for example. Thus, with regard to each sample, an array of gene measurements is taken. For example, each array may take measurements with regard to about 50,000 genes. For each gene measured, the differential values across the entire set of samples taken may be plotted to determine the response profile or response expression signature of activity across the samples taken. By looking at the trends of these response expression signature profiles, one may identify genes whose activity matches the profile or expected profile of the diseased tissue across the samples taken.

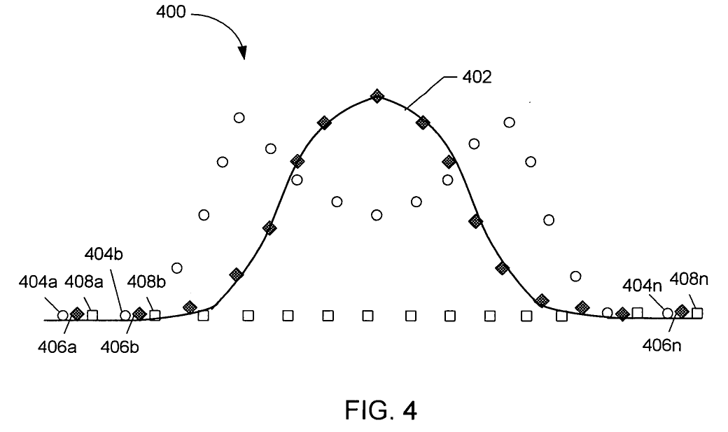

For example, FIG. 4 shows an idealized, schematic representation of a plot 400 for measured gene response expression profiles 404, 406 and 408 corresponding to three genes selected from the arrays for demonstration purposes, with a trend curve 402 of disease activity/expected disease activity along the arrow 106 in accordance with one embodiment of the present teachings. In this embodiment, each of the gene response expression profiles 404, 406 and 408 may be a normalized gene response expression profile, i.e., each profile consists of measured gene expression levels that are normalized with respect to a corresponding baseline reference signature. The baseline reference signature may be the measured gene expression levels of the reference sample 108a or 108n using the single two-color microarray, two single-color microarrays or both, as described above. In general, the trend of disease activity 402 can be determined by conceptual study that is not described in detail for simplicity.

As can be noticed, the gene response expression profile 406 “synchronizes” with the trend curve 402, which implies that the gene that is represented by gene response expression profile is related to, or involved in the disease activity. The gene corresponding to response expression profile 404 might be considered less relevant or irrelevant to the disease activity, while the gene corresponding to response expression profile 408 indicates a baseline profile and can be considered irrelevant or neutral. Thus, based on the plot 400, one can separate gene response expression profiles and distinguish gene response expression profile 406 that appears to be specific to the pure diseased cells.

In FIG. 4, only three gene response expression profiles 404, 406 and 408 are shown, for simplicity. Typically, there may be about 30,000 genes (mRNAs) in a heterogeneous tissue sample, which yields more than 30,000 gene expression profiles for each tissue sample 108a, 108b, . . . , 108n taken including functional variants. In one embodiment of the present teachings, each gene response expression profile can be compared with the trend curve 402 by fitting to a statistical regression function. In another embodiment, comparison of the trend curve 402 with each gene response expression profile can be realized by calculating a conventional p-value to test the null hypothesis between the gene expression profile and the trend curve 402. (Hereinafter, the term “p-value” refers to the significance level, or equivalently the probability that a true null hypothesis is being rejected.)

As shown in FIG. 4, the comparison has been limited only to two one-dimensional curves: the trend curve 402 and one of the gene response expression profiles 404, 406 and 408. However, in another embodiment of the present teachings, the comparison can be extended to two- or three-dimensional space. For example, in two-dimensional space, the samples taken along the entire arrows 106 can be compared with a trend surface (not shown in FIG. 4 for simplicity).

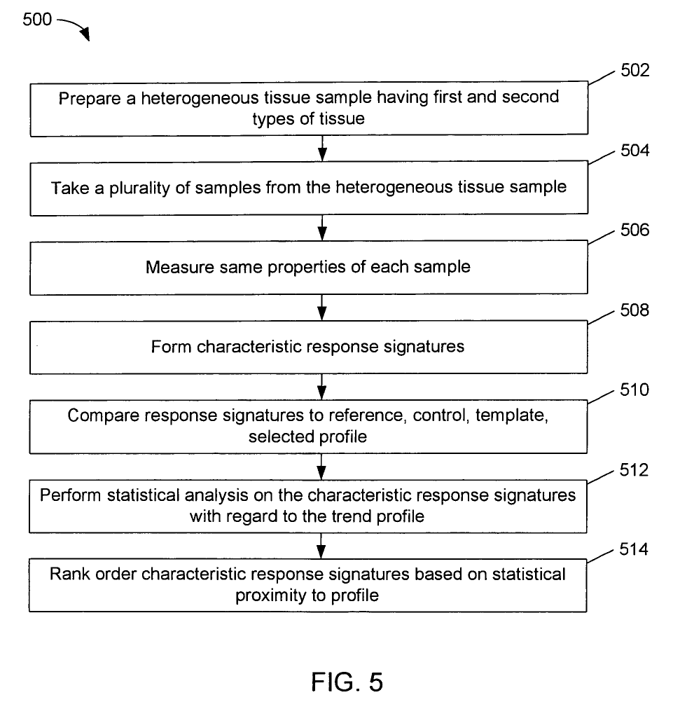

FIG. 5 shows a flow chart 500 indicating an example of steps that may be taken as an approach to identifying gene expression signatures relating to a tissue type, such as a diseased tissue, for example, in a heterogeneous tissue sample. It is noted, however, that the consideration of tissue samples is merely for exemplary purposes, as the present invention may be applied to any unknown heterogeneous mixture of substances where a property or material of interest varies in samples taken from locations across the region occupied by the mixture. In step 502, a heterogeneous sample is prepared, wherein the heterogeneous sample has a first type of tissue (such as healthy tissue) and a second type of tissue (such as diseased tissue). Next, a plurality of samples are taken from locations which can be characterized by a profile or expected profile of a characteristic of the diseased tissue, such as relative density of diseased tissue versus healthy tissue, or relative activity of the diseased tissue, for example. Typically, the sampling starts from the first type of tissue, to establish a baseline or reference, and proceeds incrementally across an identified region in which the second type of tissue is located, to an opposite boundary of the identified region, and finishes with at least one sample that is again thought to be wholly characterized by the first type of tissue. In step 506, each sample is analyzed to take measurements characterizing each sample. The measurements taken are for the same characteristics with regard to each sample. For example, gene expression levels may be measured for each sample, although the present invention is not limited to this type of analysis. Any characteristics that are measurable (quantifiable) and thought to be related to the activities (both phenotypic and genotypic activities) of the phenomenon being studied may be used in the process. For example, the process may be applied in studying phase relationships between treatment responses of diseased tissues to treatments applied thereto, using measured expression profiles of the diseased tissues as measured when untreated versus treated. Such studies are described in more detail in co-pending, commonly owned application Ser. No. 10/640,081.

Characteristic response signatures for each characteristic are then formed, at step 508, across the entirety of the samples taken, by considering the same characteristic for each sample to form a signature. The response signatures, which form profiles, are then compared to a profile or expected profile characterizing the diseased tissue (or other tissue feature being studied) at step 510. Statistical analysis is performed on the characteristic response signatures with regard to the profile or expected profile characterizing the diseased tissue (or other anomaly being studied) at step 512, to determine those response signatures that most closely conform to the profile or expected profile. The characteristic response signatures may be rank ordered at step 514, based upon their proximity to the profile or expected profile, to clearly identify those characteristic response signatures most closely involved in the phenomenon being studied. Additionally, p-values may be calculated and assigned to the characteristic response signatures, based on their proximity to the profile or expected profile.

With regard to microarray analysis, as mentioned in the earlier examples, the measured properties in step 506 are gene expression levels. Thus, at least one microarray is processed for each tissue sample to measure gene expression levels from all genes measured by the microarray. Each characteristic response signature produced in such an example includes differential expression values for the same gene across all tissue samples. Hence, a differential expression response signature is produced for each gene. The gene differential expression response signatures may be assigned p-values based upon how closely they conform to the profile or expected profile of the disease activity.

In processing the measured gene expression levels, the processing may include normalization of the measured gene expression levels with respect to a corresponding baseline reference signature.

With regard to the trend profile used to compare the response signatures to, the trend profile is typically known or hypothesized from a conceptual knowledge of the disease. The comparisons may involve comparing the trend profile with each of the differential expression response signatures using statistical analysis. In one embodiment of the present teachings, the comparison can be realized by curve fitting to a statistical regression function. In another embodiment, the comparison can be realized by calculating conventional p-values to test the null hypothesis between the processed gene expression response levels and the model trend profile of the cell activity. Based on the statistical analysis, one can separate the differential expression response signatures (profiles) of the genes and distinguish differential expression response signatures, and the genes that are associated with the response signatures, to identify those genes which are indicated as being related to or involved in the activity being studied, such as activity of a disease process.

As mentioned above, there may be more than 30,000 genes in a typical heterogeneous tissue sample and a scaled/corrected p-value for each gene can be calculated following the flow chart 500. A reliable p-value requires a sufficient population of samples taken from the heterogeneous tissue sample, where each sample may have its own mixture ratio of the two types of tissue. Another way of providing such population of samples can be mixing two types of tissue at controlled mixture ratios. For example, one can consider a series of microarrays over changing condition, e.g., the Gene Logic mixture dilution series, where the hybrid solution goes incrementally from 100% liver tissue to 100% CNS (central nervous system) cell line. As genes can be expressed differently in the two types of tissue, a p-value for each gene expression profile and the trend profile can be calculated. Then, as disclosed in one embodiment of the present teachings, the p-values can be sorted and plotted in logarithmic scale to generate a curve, which may be referred to as a “pCurve™.”

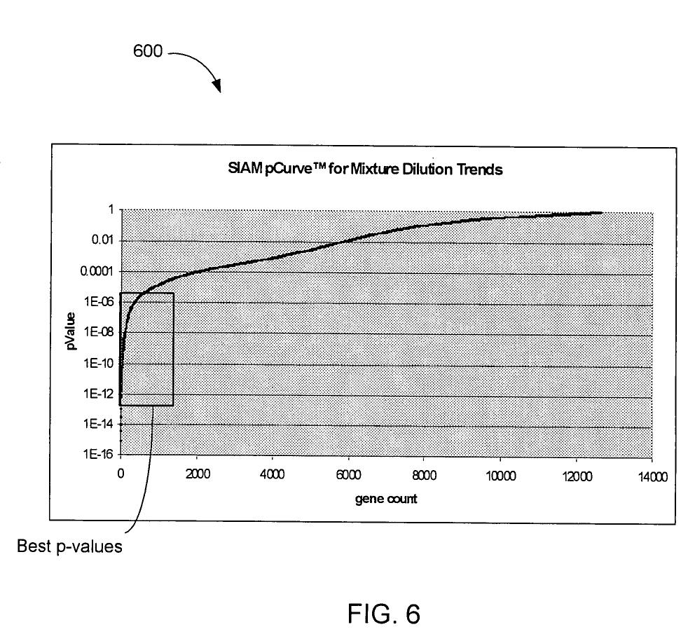

FIG. 6 is a plotted curve 600 (e.g., p-Curve) of sorted p-values against the ranks of the p-values based on the order of the sorted p-values from highly-significant, low p-values to larger, less-significant p-values. Each p-value, as statistically calculated, represents the probability that a response signature profile does not match a specified test signature profile defined by a template and/or clustering. However, a multiplicity of coincident p-values will stochastically produce some optimistic results. Hence, the smallest p-values forming the steep part of the pCurve are the most reliable. In this example, curve (pCurve) 600 is for a Gene Logic mixture dilution series of liver tissue and CNS cell line. Curve 600 can be used to identify genes behaving differently between those two types of tissue. For example, the first 6,000 genes in FIG. 6 show a “very significant difference” (or, equivalently p-value<0.01), which may imply that the first 6,000 genes are related to the CNS cell line.

Curve 600 may also be used to compare methods of signal processing and/or assays for gene expression levels. The pCurve with lowest ensemble p-values is best, e.g., the pCurve having the lowest mean-p-value, the steepest slope of plotted p-values, or greatest area above the curve, etc., may be produced to rank the two methods according to their ability to find significant effects given the design of changing conditions. For example a curve 600 for a mixture-dilution series between two dissimilar biological samples can test the relative capabilities of the two signal-processing and/or assay methods to find gene trends within both random and bias error environments. A less discriminating method would tend to have a higher flatter curve 600, relative to the curve 600 for a more discriminating method which curve would be relatively lower and steeper.

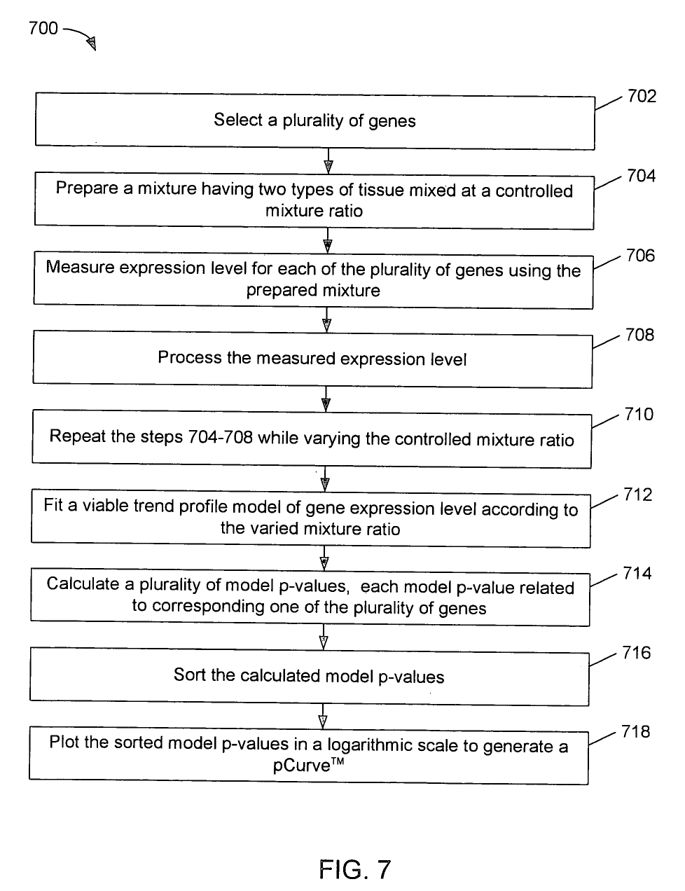

FIG. 7 is a flow chart 700 including steps for an example of validating or calibrating a curve 600 in accordance with one embodiment of the present teachings. In step 702, a plurality of genes may be selected. Next, a mixture having two types of tissue, such as liver tissue and CNS cell lines, are prepared at a controlled mixture ratio in step 704. Then, gene expression levels for each gene are measured using the prepared mixture and processed, such as by assaying to obtain microarray measurements, for example, in steps 706 and 708, respectively. Optionally, the processing may include normalization of the measured gene expression levels with respect to a reference value, wherein the reference value can be the measured gene expression level of the pure first type of tissue.

The steps 704-708 are repeated while the controlled mixture ratio is varied as shown in step 710. Then, according to the variation of the controlled mixture ratio, a viable trend profile model of gene expression level, i.e., a response profile, for both validating and templating, may be fitted in step 712. A p-value to test the null hypothesis between the processed gene expression response profiles/signatures for each gene and the fitted trend profile model is calculated in step 714. Once p-values for the plurality of genes are calculated, the p-values are sorted and plotted on a logarithmic scale to yield a curve 600 in steps 716-718.

In another embodiment of the present teachings, curve 600 may be generated by carrying out the steps 502-514 from FIG. 5 for all of the genes being measured, wherein the statistical analysis of step 512 includes calculating a p-value for each selected gene. In this embodiment, instead of varying the mixture ratio as described in step 710, a plurality of samples can be taken from a heterogeneous sample tissue at various locations as described in step 504.

In general, microarray techniques are based on the binding (hybridizing) of targets to the probes. For each probe, most of the hybridized targets have a subsequence matching to the probe, which is called “specific bonding.” However, some of the hybridized targets may have sub-sequences that mismatch partially or entirely, which is called “non-specific bonding.” Such non-specific bonding, which is a source of noise in measurements of gene expression levels, depends on the genetic environment of the mixture present in a heterogeneous tissue sample. Thus, the noise property of each probe may change from one study to another and, as a consequence, replicates of measurements may need to be performed for conventional statistical analysis. In one approach, the replicates of measurements may be performed by running multiple microarrays using the same sample, i.e., technical replicates. In another approach, each replicate includes the process of creating a sample as the noise could be in biological preparation of samples, i.e., biological replicates. Yet another approach may be that of combining the two aforementioned approaches.

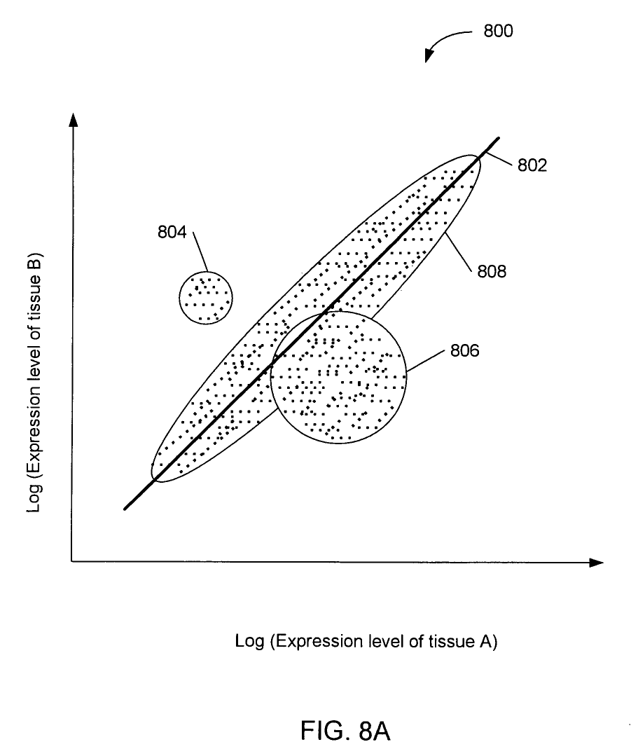

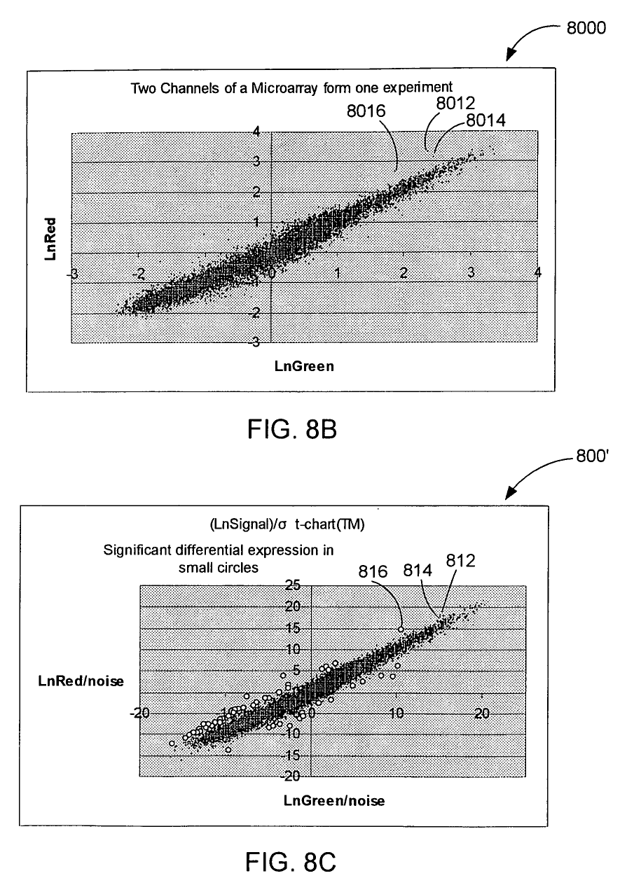

A “T-chart™” 800 (or, equivalently a scatter plot) of gene expression levels scaled by noise as obtained by replicates of measurements may be used to distinguish genes that have true differential expressions from those that might appear to be differentially expressed when plotting one value per gene, but which may not be truly differentially expressed when taking noise associated with the signal into consideration. FIG. 8A is a representation of a T-chart 800 for gene expression levels of two types of tissue, type A and B, in a logarithmic scale. Typically, one of the two types of tissue may be a reference tissue, such as healthy tissue, while the other may be a diseased tissue. Each data point of the plot 800 corresponds to one replicate of measurement for a gene.

A noise cloud 804 is shown as a pattern and comprises a collection of data points obtained by replicates of measurements for a specific gene. Since noise properties of different probes can vary, this results in various differential expression values being reported by different probes, even when measuring the same gene for the same experiment, as a replicate, for example. The diameter of the noise cloud 804 is a reflection of the noise properties of the probes used. The less noisy the group of probes is, the more consistent will be the results from each replicate measured, resulting in a relatively smaller diameter cloud. The noise cloud 806 comprises a collection of data for another gene. In FIG. 8A, only two noise clouds 804 and 806 are shown for simplicity. The ellipsoid 808 embraces a collection of noise clouds corresponding to a plurality of genes (the noise clouds are not shown therein for simplicity).

The diagonal 802 is the best location of non-expressed genes because data points for non-expressed genes would be on the diagonal if there were no noise, since their expression value is 1/1. Thus, if a noise cloud, such as the noise cloud 804, does not overlap with the diagonal 802, the corresponding gene may be significantly expressed. On the contrary, if a noise cloud, such as the noise cloud 806, overlaps with the diagonal 802, the corresponding gene may not be significantly expressed. That is, if the noise cloud overlaps the diagonal by a statistically significant amount, as determined by the conventional and well-known T-statistic, for example, it would be determined that the particular gene is not expressed, e.g., in this case, not significantly down-regulated. For example, a gene may be determined to be differentially expressed when, for a p-value of 0.05, less than five percent of the noise cloud crosses over diagonal 802.

The gene corresponding to the noise cloud 806 does appear “down-regulated,” since the center of cloud 806 is below the diagonal 802. However, it is quite likely that the gene may not be down-regulated due to its large noise level relative to its significance level.

FIGS. 8B-8C show a comparison between a plot 8000 (FIG. 8B) of gene expression levels from a red channel (LnRed) of a two-channel microarray platform plotted against gene expression levels for the same genes on a green channel (LnGreen) in a logarithmic scale. Chart 800′ (FIG. 8C) shows a T-chart of the same data, after noise-normalizing the data in the manner described above. The data points that are lighter in shade are those that were determined to be differentiated. Thus, in comparing these charts, it can be observed that some of the data points which might appear to show differentiated genes (e.g., 8012, 8014) are actually determined to not be significantly differentiated (e.g., 812, 814) when accounting for noise factors. In contrast, data point 8016 appears to be differentiated, and is also determined to be differentiated (816) after accounting for noise factors.

As mentioned, T-chart 800 is presented in a logarithmic scale. In a typical assay of biological study, the gene expression levels are generally plotted in logarithmic scale for both statistical and biological reasons. From a statistical standpoint, noise levels are usually approximately proportional to the signal level magnitudes. By taking the log of the readings, this homogenizes the noise levels relative to the signals, so that signal levels are not skewed by proportional log levels. From a biological viewpoint, the log of the signal is often proportional to the log of the stimulus, such as for example in the cases of vision, sound, and/or treatment versus response phenomena.

The T-chart 800 in FIG. 8A can be extended to high-dimensional space when gene expression levels are measured using multi-microarray apparatus. Based on the same reasoning applied to the analysis of gene expression in the T-chart 800, a gene corresponding to a noise cloud in high-dimension space may be significantly expressed if the noise cloud does not overlap the high-dimensional diagonal.

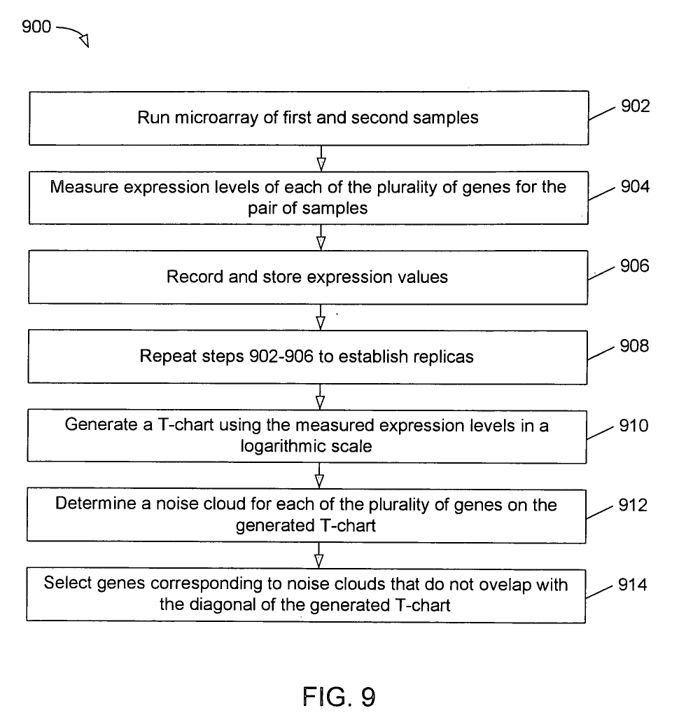

FIG. 9 is a flow chart 900 for steps to distinguish differentially-expressed genes by preparing replicates and using the techniques described above with regard to FIG. 8A. The flow chart in FIG. 9 describes a comparison of two channels, but this method is not limited thereto, as multi-channel, multi-dimensional analysis may be similarly carried out. At step 902, a first sample is processed on a microarray, and a second sample is processed on a second microarray, or the second channel/color of a first microarray. The second sample may be a reference to be used in calculating differential expressions by comparison with the first sample. At step 904, expression values from each of the probes with regard to the at least first and second samples are determined, and these expression values are recorded or stored at step 906. The steps 902-906 are repeated until a sufficient number of replicates of measurements are performed at step 908. Typically four or five replicates (degrees of freedom) produce adequate statistical leverage to estimate the noise cloud. In the case where one of the channels is a reference, and a two single channel microarray technique is used, the repetition of the steps to form the replicates does not require re-processing the reference channel, as this may be used for comparison against all the replicates that are processed. Alternatively, a universal reference may be prepared once until supplies dwindle, in which case another universal reference is produced. The two universal references are matched as close as possible, but may not be identical. Noiseless correction factors between the new and old reference are easily established by replicate comparisons between them using microarray technology. These methods are not limited by platform type, as single color (single channel) or dual channel (dual color) platforms may be employed.

At step 910, a T-chart is generated, preferably in a logarithmic scale, using the measured and stored gene expression levels, in the manner described with regard to FIG. 8A above. Then, noise clouds generated from the plotting in step 910 are observed for each gene of interest, at step 912. Optionally, a forty-five degree diagonal line may be overlaid on the T-chart 800 to aid in visibly determining whether any particular noise cloud is distinctly separated from the housekeeping genes (i.e., those genes substantially aligned with the forty-five degree diagonal which are considered to be neutral or not expressed). By observation or other analysis of the T-chart 800, those genes corresponding to noise clouds that do not overlap with the diagonal of the T-chart 800 are selected or identified as differentially-expressed genes in step 914. Optionally, the location of each point can be scaled by its particular noise factors to produce a chart of “standardized” points. The distance of each point from the diagonal becomes multiples of its particular noise factors. Hence, the distance automatically infers degree of overlap of noise with the diagonal, eliminating any need for plotting noise clouds.

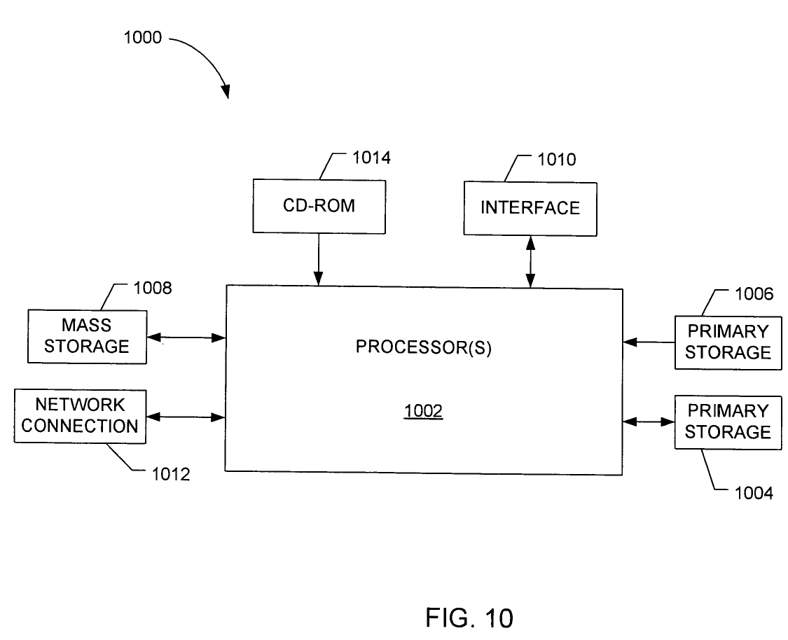

FIG. 10 illustrates a typical computer system in accordance with an embodiment of the present invention. The computer system 1000 includes any number of processors 1002 that are coupled to storage devices including primary storages 1004 and 1006. As is well known in the art, primary storage 1006 acts to transfer data and instructions uni-directionally to the CPU and primary storage 1004 is used typically to transfer data and instructions in a bi-directional manner. Both of these primary storage devices may include any suitable computer-readable media. A mass storage device 1008 is also coupled bi-directionally to CPU 1002 and provides additional data storage capacity and may include any of the computer-readable media. Mass storage devices 1008 may be used to store programs, data and the line and is typically a secondary storage medium such as a hard disk that is slower than primary storage. It will be appreciated that the information retained within the mass storage device 1008, may, in appropriate cases, be incorporated in standard fashion as part of primary storage 1006 as virtual memory. A specific mass storage device such as CD-ROM 1014 may also pass data uni-directionally to the CPU.

CPU 1002 is also coupled to an interface 1010 that includes one of more input/output devices such as video monitors, track balls, mice, keyboards, microphones, touch-sensitive displays, transducer card readers, magnetic or paper tape readers, tablets, styluses, voice or handwriting recognizers, or other well-known input devices such as, of course, other computers. Finally, CPU 1002 optionally may be coupled to a computer or telecommunications network using a network connection as shown generally at 1012. With such a network connection, it is contemplated that the CPU might receive information from the network, or might output information to the network in the course of performing the above-described method steps. The above-described devices and materials will be familiar to those of skill in the computer hardware and software arts.

The hardware elements described above may implement the instructions of multiple software modules for performing the operations of this invention. In addition, embodiments of the present invention further relate to computer readable media or computer program products that include program instructions and/or data for performing various computer-implemented operations. The media and program instructions may be those specially designed and constructed for the purposed of the present invention, or they may be of the kind well known and available to those having skill in the computer software arts. Examples of computer-readable media includes, but not limited to, magnetic media such as hard disks, floppy disks, and magnetic tape; optical media such as CD-ROM, CDRW, DVD-ROM, or DVD-RW disks; magneto-optical media such as floppy disks, and hardware devices that are specially configured to store and perform program instructions, such as read-only memory devices (ROM) and random access memory (RAM). Examples of program instructions include both machine codes, such as produced by a computer, and files containing higher level codes that may be executed by the computer using an interpreter.

While the present invention has been described with reference to the specific embodiments thereof, it should be understood, of course, that the foregoing relates to preferred embodiments of the invention and that modifications may be made without departing from the spirit and scope of the invention as set forth in the following claims.

In addition, many modifications may be made to adapt a particular situation, treatment, tissue sample, process, process step or steps, to the objective, sprit and scope of the present invention. All such modifications are intended to be within the scope of the claims appended hereto.

1. A method for rank ordering characteristic signatures of cell properties, said method comprising the steps of:
forming a plurality of characteristic signatures for a plurality of cell properties having been measured from a plurality of samples taken from a heterogeneous tissue region, wherein the heterogeneous tissue region includes a first portion having at least first and second types of tissue, bordered by a second portion, said second portion considered to be devoid of the second type of tissue, wherein the plurality of samples have been taken from successive locations along a determined profile of locations through the heterogeneous tissue region, with at least one sample being taken from the second portion, and wherein each of said characteristic signatures characterizing one of the plurality of properties, respectively;
providing a trend profile of cell activity for the second type of tissue along the determined profile of locations through the heterogeneous tissue region;
performing statistical analysis on each of the plurality of characteristic signatures with regard to the provided trend profile; and
rank ordering the plurality of characteristic signatures based on proximity to the trend profile as determined by the statistical analysis.

2. The method of claim 1, further comprising the step of:
measuring the plurality of cell properties for each of the plurality of samples.

3. The method of claim 1, further comprising the steps of:
providing the heterogeneous tissue region: and
taking the plurality of samples from the heterogeneous tissue region.

4. The method of claim 3, further comprising the step of:
measuring the plurality of cell properties for each of the plurality of samples.

5. The method of claim 1, wherein the step of forming a plurality of characteristic signatures includes normalizing each of the plurality of characteristic signatures with respect to a baseline reference signature, said baseline reference signature corresponding to a measured property of a sample taken from the second portion.

6. The method of claim 1, wherein the step of performing statistical analysis includes:
comparing each of the plurality of characteristic signatures with the provided trend profile by curve-fitting to a statistical regression function, wherein said curve-fitting determines the degree of proximity of each of the plurality of characteristic signatures to the provided trend profile.

7. The method of claim 1, wherein the step of performing statistical analysis includes:
calculating a p-value with regard to each of the plurality of characteristic signatures, to test the null hypothesis between each of the plurality of characteristic signatures and the provided trend profile.

8. The method of claim 1, wherein the step of performing statistical analysis is done in one-, two- or three-dimensional space.

9. The method of claim 1, wherein the first type of tissue is healthy tissue.

10. The method of claim 1, wherein the second type of tissue is diseased tissue.

11. The method of claim 1, wherein one of the plurality of properties is an expression level of a gene.

12. The method of claim 2, wherein the step of measuring a plurality of properties includes:
processing each of the plurality of samples using a microarray technique.

13. The method of claim 2, wherein the step of measuring a plurality of properties includes:
processing each of the plurality of samples on a single two-color microarray, two single-color microarrays or both.

14. A method comprising forwarding a result obtained from the method of claim 1 to a remote location.

15. A method comprising transmitting data representing a result obtained from the method of claim 1 to a remote location.

16. A method comprising receiving a result obtained from a method of claim 1 from a remote location.

17. A computer readable medium carrying one or more sequences of instructions for rank ordering characteristic signatures of cell properties measured from a plurality of samples taken from a heterogeneous region, wherein a first portion of the heterogeneous tissue region has at least first and second types of tissue and is bordered by a second portion of the heterogeneous tissue region, wherein the second portion is considered to be devoid of the second type of tissue, and wherein the plurality of samples have been taken from successive locations along a determined profile of locations through the heterogeneous tissue region, with at least one sample being taken from the second portion, wherein execution of one or more sequences of instructions by one or more processors causes the one or more processors to perform the steps of:
forming a plurality of characteristic signatures using the measured plurality of properties, each of said characteristic signatures characterizing one of the plurality of properties, respectively;
providing a trend profile of cell activity for the second type of tissue along the determined profile of locations through the heterogeneous tissue region;
performing statistical analysis on each of the plurality of characteristic signatures with regard to the provided trend profile; and
rank ordering the plurality of characteristic signatures based on proximity to the trend profile as determined by the statistical analysis.

18. A system for rank ordering characteristic signatures of cell properties generated from tissue samples taken from a heterogeneous tissue region, wherein a first portion of the heterogeneous tissue region has at least first and second types of tissue and is bordered by a second portion of the heterogeneous tissue region, wherein the second portion is considered to be devoid of the second type of tissue, the system comprising:
means for providing a trend profile of cell activity for the second type of tissue along a determined profile of locations through the heterogeneous tissue region from which tissues samples are taken as the sources of the characteristic signatures;
means for performing statistical analysis on each of the plurality of characteristic signatures with regard to the provided trend profile; and
means for rank ordering the plurality of characteristic signatures based on proximity to the trend profile as determined by the statistical analysis.

19. The system of claim 18, further comprising
means for forming the plurality of characteristic signatures based on measurements of a plurality of properties characteristic of the tissues, each of said characteristic signatures related to a corresponding one of the plurality of properties.

20. The system of claim 18, further comprising:
means for measuring the plurality of properties for each of the plurality of samples.

21. A method for validating or calibrating a plotted curve of sorted p-values against the ranks of the p-values based on the order of the sorted p-values, wherein the p-values are calculated with regard to characteristic signature profiles each generated from a plurality of property values from a plurality of samples, and wherein each said p-value, as statistically calculated, represents the probability that the corresponding characteristic signature profile does not match a predefined signature profile, said method comprising the steps of:
selecting a plurality of characteristics from a set of characteristic properties from the samples;
preparing a sample as a mixture having two types of tissue mixed at a controlled mixture ratio;
measuring the selected characteristics in the prepared mixture;
repeating said preparing and measuring steps, while varying the controlled mixture ratio with each repetition of said preparing and measuring steps;
generating a trend profile model based on the controlled variations in the mixture ratios;
calculating a plurality of model p-values, each model p-value generated based on a comparison between a characteristic response signature, generated from characteristic values of one of the selected characteristics across all samples, with the trend profile model;
sorting the calculated model p-values; and
plotting the sorted model p-values against the ranks of the sorted p-values, based on the order of the sorted p-values.

22. The method of claim 21, wherein said model p-values are plotted in a logarithmic scale

23. The method of claim 21, wherein the step of preparing a mixture comprises picking a sample from a heterogeneous tissue sample having the two types of tissue.

24. The method of claim 21, wherein the characteristics are gene expression levels, said gene expression levels being processed to form said characteristic signatures comprising gene expression response signatures.

25. The method of claim 24, wherein the measured expression levels are further processed to normalize the measured expression levels with respect to a corresponding baseline reference signature, said corresponding baseline reference signature being a measured gene expression level of one of the two types of tissue.

26. A computer readable medium carrying one or more sequences of instructions for validating or calibrating a plotted curve of sorted p-values against the ranks of the p-values based on the order of the sorted p-values, wherein the p-values are calculated with regard to characteristic signature profiles each generated from a plurality of property values from a plurality of samples, and wherein each said p-value, as statistically calculated, represents the probability that the corresponding characteristic signature profile does not match a predefined signature profile, wherein execution of one or more sequences of instructions by one or more processors causes the one or more processors to perform the steps of:
selecting a plurality of characteristics from a set of characteristic properties from the samples;
preparing a sample as a mixture having two types of tissue mixed at a controlled mixture ratio;
measuring the selected characteristics in the prepared mixture;
repeating said preparing and measuring steps, while varying the controlled mixture ratio with each repetition of said preparing and measuring steps;
generating a trend profile model based on the controlled variations in mixture ratio;
calculating a plurality of model p-values, each model p-value generated based on a comparison between a characteristic response signature, generated from characteristic values of one of the selected characteristics across all samples, with the trend profile model;
sorting the calculated model p-values; and
plotting the sorted model p-values against the ranks of the sorted p-values, based on the order of the sorted p-values.

27. A system for validating or calibrating a plotted curve of sorted p-values against the ranks of the p-values based on the order of the sorted p-values, wherein the p-values are calculated with regard to characteristic signature profiles each generated from a plurality of property values from a plurality of samples, and wherein each said p-value, as statistically calculated, represents the probability that the corresponding characteristic signature profile does not match a predefined signature profile, the system comprising:
means for selecting a plurality of characteristics from a set of characteristic properties from the samples;
means for preparing a sample as a mixture having two types of tissue mixed at a controlled mixture ratio;
means for measuring the selected characteristics in the prepared mixture;
means for repeating said preparing and measuring steps, while varying the controlled mixture ratio with each repetition of said preparing and measuring steps;
means for generating a trend profile model based on the controlled variations in mixture ratio;
means for calculating a plurality of model p-values, each model p-value generated based on a comparison between a characteristic response signature, generated from characteristic values of one of the selected characteristics across all samples, with the trend profile model;
means for sorting the calculated model p-values; and
means for plotting the sorted model p-values against the ranks of the sorted p-values, based on the order of the sorted p-values.

28. A method for distinguishing differentially-expressed genes based on plotting one set of expression level values against another set of corresponding expression level values, the method comprising the steps of:
measuring an expression level for each of one or more genes for first and second samples, respectively;
plotting the measured expression levels for the first sample against the measured expression levels for the second sample;
repeating said measuring and plotting steps to establish a number of replicates of the measured expression levels;
determining whether a particular gene from a first sample is differentially expressed relative to the same gene from the second sample, based upon the values of the measured expression levels and their replicates for the particular gene.

29. The method of claim 28, wherein said determining is based on a noise cloud generated by plotting the measured expression level and its replicates with regard to the particular gene in the first sample, against the measured expression level and its replicates with regard to the particular gene in the second sample, wherein the particular gene is determined to be differentially expressed when said less than a predefined percentage of said noise cloud intersects a line representing neutral genes.

30. The method of claim 29, wherein said predefined percentage is five percent at a p-value of 0.05.

31. The method of claim 28, wherein said determining is based on scaling the measured expression level of the particular gene in each of the first and second samples by noise factors characterized by the respective replicates to produce standardized expression levels for the particular gene with regard to the first and second samples, wherein the particular gene is determined to be differentially expressed when said standardized expression levels are plotted as a distance from a line representing neutral genes that represents a p-value of about 0.05 or less.

32. The method of claim 28, carried out in multi-dimensional space with regard to greater than two samples.

33. A computer readable medium carrying one or more sequences of instructions for distinguishing differentially-expressed genes based on a distinguishing differentially-expressed genes based on plotting replicates of expression level values against corresponding replicates of another set of expression level values, wherein execution of one or more sequences of instructions by one or more processors causes the one or more processors to perform the steps of:
plotting an expression level of each of one or more genes for a first sample against an expression level for each of the same one or more genes in a second sample;
plotting one or more replicates of said expression levels; and
determining whether a particular gene from a first sample is differentially expressed relative to the same gene from the second sample, based upon the values of the measured expression levels and their replicates for the particular gene.

33. A system for distinguishing differentially-expressed genes based on plotting one set of expression level values against another set of corresponding expression level values, the system comprising:
means for plotting an expression level of each of one or more genes for a first sample against an expression level for each of the same one or more genes in a second sample;
means for plotting one or more replicates of said expression levels; and
means for determining whether a particular gene from a first sample is differentially expressed relative to the same gene from the second sample, based upon the values of the measured expression levels and their replicates for the particular gene.
